(19) United States
(12) Patent Application Publication
(10) Pub. No.: US 2005/0255071 A1
(43) Pub. Date: Nov. 17, 2005

(54) Preparation having improved therapeutic breadth comprising nucleotide synthesis inhibitors

(75) Inventors: Jurgen Lindner, Marburg (DE); Burkhard Haase, Hofheim (DE)

Correspondence Address: SYNNESTVEDT & LECHNER LLP, 2600 ARAMARK TOWER, 1101 MARKET STREET, PHILADELPHIA, PA, 19107-2950, US

(73) Assignee: Aventis Pharma Deutschland GmbH, Frankfurt Am Main (DE)

(21) Appl. No.: 11/178,987

(22) Filed: Jul. 11, 2005

Related U.S. Application Data
(63) Continuation of application No. 09/457,596, filed on Dec. 9, 1999, now abandoned.

(30) Foreign Application Priority Data
Dec. 10, 1998 (DE) 198 07 009.0

(51) Int. Cl. A61K 38/13; A61K 31/785; A61K 31/537; A61K 31/4439; A61K 31/42; A61K 31/277; A61K 33/44

(52) U.S. Cl. 424/70.17; 424/125; 514/352; 514/378; 514/340; 514/521; 514/234.2; 514/11

(57) ABSTRACT

A preparation comprising a compound which essentially prevents the enterohepatic circulation of nucleotide synthesis inhibitors or antagonizes the action of the nucleotide synthesis inhibitors with a displacement in time, and a nucleotide synthesis inhibitor such as brequinar, mycophenolatemofetil (2-morpholinoethyl (E)-6-(1,3-dihydro-4-hydroxy-6-methoxy-7-methyl-3-oxoisobenzofuran-5-yl)-4-methyl-4-hexenoate), methotrexate, mizoribine and compounds of formulae (I) and (II) 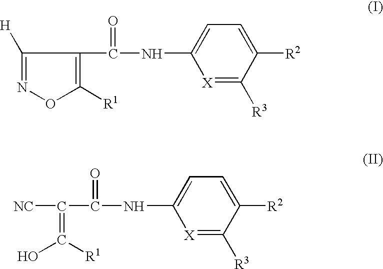 is suitable for the treatment of immunological disorders, cancer, or in transplantations.

### CROSS-REFERENCE TO RELATED APPLICATIONS

This application is a Continuation of U.S. patent application Ser. No. 09/457,596 filed Dec. 9, 1999, which claims priority under 35 U.S.C. § 119 to German Application No. 198 57 009.0 filed Dec. 10, 1998, the disclosures of which are incorporated herein by reference.

### BACKGROUND OF THE INVENTION

Strongly basic anion exchangers are employed therapeutically as hypolipidemics in heterozygous familial hypercholesterolemia and other primary hyperlipoproteinemias having a principal proliferation of the LDL fraction or in chologenic diarrheas. Examples of suitable active substances which are employed as hypolipidemics are N-(2-aminoethyl)-N′-[2-[(2-aminoethyl)amino]ethyl]-1,2-ethanediamine polymers with (chloromethyl)oxirane, which is also called colestipol (COLESTID®) or colestyramine (CAS-No. 11 041-12-6), which is a styrene/divinylbenzene copolymer. Isoxazole or crotonamide derivatives are described in, for example, EP 484 223, EP 529 500, U.S. Pat. No. 4,061,767, EP 538 783, and EP 551 230.

Compounds which inhibit purine or pyrimidine synthesis are called nucleotide synthesis inhibitors (Burkhardt and Kalden, Rheumat. Int. (1997), 17:85-90). Examples are compounds of formula (1) and/or (II), brequinar (6-flouro-2-(2′-fluoro[1,1′biphenyl]-4-yl)-3-methyl-4-quinolinecarboxylic acid), mycophenolatemofetil (2-morpholinoethyl (E)-6-(1,3-dihydro-4-hydroxy-6-methoxy-7-methyl-3-oxoisobenzofuran-5-yl)-4-methyl-4-hexenoate), methotrexate (CAS No. 59-05-02), or mizoribine (CAS No. 50924-49-7). Such compounds are absorbed in the intestine of patients after oral administration (absorption peak), lead to constant high blood levels. These nucleotide synthesis inhibitors are excreted again in the intestine via the liver and in bile. The excreted compounds can then partially be absorbed again from the intestine and secreted into the blood. The compounds mentioned are therefore subject to enterohepatic circulation.

### SUMMARY OF THE INVENTION

In the employment of nucleotide synthesis inhibitors for affecting the immune system, it was surprisingly found that only brief active effects of these substances are needed for the desired action on the immune system. If blood levels of these substances which lead to active effects are maintained over a relatively long period, although the side effects increase, the desired action on the immune system is not increased. By limiting the active effects to a short period of time, the tolerability of a therapy can be improved while maintaining the desired pharmacodynamic effects on the immune system (resulting in improved therapeutic breadth).

In the case of nucleotide synthesis inhibitors subject to enterohepatic circulation, the duration of action can be reduced by administering substances which interrupt enterohepatic circulation. Owing to interruption of the enterohepatic circulation, the desired action on the immune system is maintained, but the side effects are drastically reduced. The above-mentioned nucleotide synthesis inhibitors can also have an improved therapeutic breadth in their action if compounds which antagonize the action of the nucleotide synthesis inhibitors are administered with a displacement in time—i.e., later than the nucleotide synthesis inhibitors.

The term therapeutic breadth is understood here as a measure of the tolerability of a pharmaceutical compound, composition, or preparation, and is essentially the difference between the lowest dose which still provides the desired therapeutic effects, and the dose which leads to side effects. Yardsticks for the improvements achieved are the amount of red blood corpuscles, the hemoglobin content, the hematocrit, the amount of glutamate oxaloacetate transaminase, glutamate pyruvate transaminase, alkaline phosphatase (from bone marrow) or amylase, and the weight, in comparison with untreated patients.

The invention therefore relates to a preparation comprising:
1) at least one compound which prevents the enterohepatic circulation of the nucleotide synthesis inhibitors, or antagonizes the action of the nucleotide synthesis inhibitors with a displacement in time, and
2) at least one nucleotide synthesis inhibitor selected from brequinar, mycophenolatemofetil (2-morpholinoethyl (E)-6-(1,3-dihydro-4-hydroxy-6-methoxy-7-methyl-3-oxoisobenzofuran-5-yl)-4-methyl-4-hexenoate), methotrexate, mizoribine, and compounds of formulae (I) and (II) 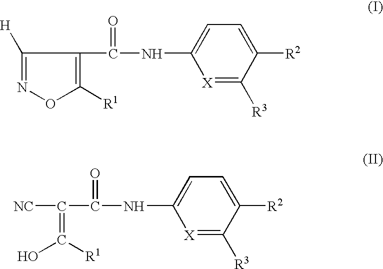
and/or an optionally stereoisomeric form of the compound of formula (I) and (II) and/or a physiologically tolerable salt of the compound of formula (II), where
R¹ is
   a) —(C₁-C₄)-alkyl,
   b) —(C₃-C₅)-cycloalkyl,
   c) —(C₂-C₆)-alkenyl, or
   d) —(C₂-C₆)-alkynyl;
R² is
   a) —CF₃,
   b) —O—CF₃,
   c) —S—CF₃,
   d) —OH,
   e) —NO₂,
   f) halogen,
   g) benzyl,
   h) phenyl,
   i) —O-phenyl, which is unsubstituted,
   j) —CN, or
   k) —O-phenyl, which is mono- or polysubstituted by a radical independently
   Selected from
      1) (C₁-C₄)-alkyl,
      2) halogen,
      3) —O—CF₃, and
      4) —O—CH₃;
R³ is
   a) —(C₁-C₄)-alkyl,
   b) halogen, or
   c) a hydrogen atom; and
X is
   a) a-CH group, or
   b) a nitrogen atom.

A mixture of the nucleotide synthesis inhibitors and compounds of formulae (I) and (II) or salts of the compounds of formula (II) and a mixture of the compounds which essentially prevent the enterohepatic circulation of the compound of formula (I) and (II) can also be employed.

### DETAILED DESCRIPTION OF THE PREFERRED EMBODIMENTS

The term “compound which essentially prevents the enterohepatic circulation of the compound of formula (I) and (II)” is understood as meaning, for example, strongly basic anion exchangers such as colestipol and colestyramine or activated carbon. The term “compounds which antagonize the action of the nucleotide synthesis inhibitors with a displacement in time” are understood as meaning compounds such as uridine, purine, purine nucleotides or pyramidine nucleotides.

In one embodiment, a compound of formula (I) and/or (II) and/or an optionally stereoisomeric form of the compound of formula (I) or (II) and/or a salt of the compound of formula (II) is used wherein:
R¹ is
   a) methyl,
   b) cyclopropyl, or
   c) —(C₃-C₅)-alkynyl;
R² is —CF₃, or —CN;
R³ is a hydrogen atom, or methyl; and
X is a —CH group,
   In combination with at least one compound selected from aolestipol, colestyramine and activated carbon.

Preparations containing N-(4-trifluoromethylphenyl)-5-methylisoxazole-4-carboxamide, N-(4-trifluoromethylphenyl)-2-cyano-3-hydroxyhept-2-en-6-ynecarbox-amide, in combination with colestyramine, are useful.

Compounds of formula (I) and (II) are prepared according to known processes such as are described in EP 484 223, EP 529 500, U.S. Pat. No. 4,061,7667, EP 538,783, or EP 551 230. The starting substances for the chemical reactions are known, or can easily be prepared by literature methods.

The terms alkyl, alkenyl and alkynyl are understood as meaning radicals wherein the carbon chain can be straight or branched. The alkenyl or alkynyl radicals can furthermore also contain a number of double bonds or a number of triple bonds. Cyclic alkyl radicals are, for example, 3- to 5-membered monocyclic systems such as cyclopropyl, cyclobutyl, or cyclopentyl. Salts of the compound of formula (II) are, for example, sodium or lysinium salts which can be prepared as described in European Patent Applciation No. EP 0769296.

The preparation according to the invention is suitable, for example, for the treatment of:
immunological disorders;
inflammatory and cytotoxic processes in connection with gene therapy interventions;
carcinomatous disorders such as lung cancer, leukemia, ovarian cancer, sarcoma, Kaposi's sarcoma, meningioma, intestinal cancer, lymph node cancer, brain tumors, breast cancer, pancreatic cancer, prostate cancer, or skin cancer;
autoimmune disorders such as systemic lupus erythematosus, or multiple sclerosis;
rheumatic disorders;
transplantations, graft-versus-host reactions, or host-versus-graft reactions;
disorders which are caused by strongly proliferating cells;
psoriasis, or atypic dermatitis;
allergy, asthma, urticaria, rhinitis, or uveitis;
type II diabetes;
cystic fibrosis, colitis, liver fibrosis, or sepsis; and
chronic inflammatory disorders such as arteriosclerosis, Crohn's disease, ulcerative colitis.

The invention also relates to a pharmaceutical composition utilizing the inventive preparation, which comprises bringing the nucleotide synthesis inhibitors and a compound which essentially prevents the enterohepatic circulation of the nucleotide synthesis inhibitors, or antagonizes the action of the nucleotide synthesis inhibitors with a displacement in time, into a suitable administration form by combining the preparation with a pharmaceutically suitable and physiologically acceptable vehicle and, if appropriate, further suitable active compounds additives, or excipients.

The preparation according to the invention can also include compositions or combination packs in which the constituents are placed next to one another and can therefore be used simultaneously, separately, or sequentially on one and the same human or animal body. The sequential administration of a compound of formula (I) and/or (II) before the administration of a compound, which essentially prevents the enterohepatic circulation of a compound of formula (I) and (II), is preferred. To this end, for example, N-(4-trifluoromethylphenyl)-2-cyano-3-hydroxycrotonamide (called compound 1 in the following) is administered first. Colestyramine, which essentially prevents the enterohepatic circulation of compound 1, is administered with a displacement in time, i.e., 2 hours or 4 hours after the administration of compound 1. Because of the time-displaced administration of compound 1 relative to colestyramine, compound 1 is initially absorbed unhindered from the digestive tract. After the administration of colestyramine, which is not absorbed systemically, compound 1 excreted via the bowel is bound to colestyramine, and cannot therefore be reabsorbed again; as a result, an interruption to the enterohepatic circulation is brought about. Because of this process, the duration of action and the blood level of compound 1 are drastically reduced. Despite this drastically reduced blood level, the activity in a pathological animal model, such as adjuvant arthritis, is not reduced by the administration of colestyramine at a low, still just active dose of approximately 2.5 mg/kg/day of compound 1. If high doses of 25 mg/kg/day of compound 1, which already lead to various side effects are employed in the same animal model, a clear reduction in the side effects with retention of the desired actions on the immune system is observed by means of administration of colestyramine.

A preparation according to the invention can be present as a dose unit in the form of pharmaceutical forms such as capsules (including microcapsules, which in general contain no pharmaceutical vehicles), tablets including coated tablets and pills, or suppositories. It is possible, when using capsules, for the capsule material to assume the function of the vehicle and for the contents to be present, for example, as a powder, gel, solution, emulsion or dispersion. It is particularly advantageous and simple, however, to prepare oral or peroral formulations which contain the calculated amounts of the active compounds, together with any desired pharmaceutical vehicles using two active compound components, such as, 1) colestyramine, and 2) compound(s) of formula (1) and/or (II). An appropriate formulation for rectal therapy, i.e. suppositories, can also be used.

Transdermal administration in the form of ointments or creams, parenteral (intraperitoneal, subcutaneous, intramuscular) injection, or oral administration of solutions which contain the combinations according to the invention is likewise possible. In addition to the active compound, ointments, pastes, creams and powders can contain the customary vehicles, e.g., animal and vegetable fats, waxes, paraffins, starch, tragacanth, cellulose derivatives, polyethylene glycols, silicones, silicic acid, aluminum hydroxide, talc, zinc oxide, lactose, bentonites, calcium silicate, or polyamide powder, or mixtures of these substances.

Tablets, pills or granule bodies can be produced by processes such as pressing, dipping fluidized-bed processes, or pan coating, and contain vehicles and other customary excipients such as gelatin, agarose, starch (e.g. potato, corn, or wheat starch), celluloses, such as ethylcellulose, silica, magnesium carbonate, various sugars such as lactose, and/or calcium phosphates. The coating solution usually consists of sugar and/or starch syrup and generally additionally contains gelatin, synthetic cellulose esters, gum Arabic, polyvinylpyrrolidone, pigments, surface-active substances, plasticizers and similar additives known in the prior art. For the production of a pharmaceutical composition containing the preparation, any customary flow-regulating agent, lubricant or glidant such as magnesium stearate and release agents can be used. The preparations, preferably have the form of coating/core tablets or multilayer tablets, the active component 2 being in the coating or in the core or in one layer, while the active component 1 is in the core, in the coating or in another layer. The active compound components can also be present in delayed-release form, or absorbed on release-delaying material or included in the release-delaying material (e.g., those based on cellulose or polystyrene resins, such as hydroxyethylcellulose). Delayed release of the active compounds can also be achieved by providing the layer, or the relevant compartment, with customary enteric coatings.

One embodiment is a delayed release compound which essentially prevents the enterohepatic circulation of a compound of formula (I) and (II).

The dose to be used is, of course, dependent on various factors such as the living being to be treated (i.e. human or animal), its age, weight, and general state of health, the degree of severity of the symptoms, the disorder to be treated, possible concomitant disorders (if present), the nature of the concomitant treatment with other pharmaceuticals, or the frequency of the treatment or treatments. The doses are in general administered several times per day, preferably one to three times per day. The amount of each individual active compound used is based here on the recommended daily dose of the respective individual active compound and, in the combination preparation, should, in general, be from 10% to 300% of the recommended daily dose, preferably from 50% to 150%, in particular 80%. Suitable therapy with the combinations according to the invention thus consists, for example, in the administration of 1, 2, or 3 individual doses of a preparation containing N-(4-trifluoromethylphenyl)-5-methylisoxazole-4-carboxamide or N-(4-trifluromethylphenyl)-2-cyano-3-hydroxycrotonamide in an amount of from 2 mg to 250 mg, preferably 5 mg to 150 mg, in particular 10 mg to 50 mg, particularly preferably 10 mg to 20 mg; and colestyramine in an amount of from 250 mg to 6000 mg, in particular from 1500 mg to 3000 mg.

Preparations according to the invention can also be employed together with other suitable active compounds, for example antiuricopathics, analgesics, steroidal or nonsteriodal antiinflammatories, platelet aggregation inhibitors, cytokines, cytokine agonists, cytokine antagonists, or immunosuppressant compounds such as cyclosporine A, FK 506, or rapamycin.

### EXAMPLE 1

### Adjuvant-Induced Arthritis, Modification According to Perper (Proc. Soc. Exp. Biol. Med. 137, 506 (1971)).

The experimental animals used were male rats of a Lewis strain (Moellegard, Denmark) having a body weight of from 160 to 210 g. On the 1st day, the animals were injected subcutaneously, into the tail root, with complete Freund's adjuvant containing a Mycobacterium butyricum suspension in heavy paraffin oil (Difco, 6 mg/kg in paraffin oil, Merck). The compounds N-(4-trifluoromethylphenyl)-2-cyano-3-hydroxycrotonamide and colestyramine were suspended in carboxymethylcellulose (1% in water), and administered orally. The compounds were administered once daily from the 1st to the 17th day of the experiment; the paw volume and arthritis index were then determined on the 18th day.

The severity of the disorder was determined by measuring the paw volume of both hind paws. The measurement was carried out by means of the water displacement method using a 2060 Plethys monitor (Rhema-Labortechnik, Hofheim, Germany). The arthritis index was furthermore determined in the 18th day after injection.

Determination of the arthritis index was scored as follows:

| | |
|---|---|
| 1. ears | 0.5 points for each ear on which reddening occurs |
| | and nodules are formed |
| 2. nose | 1 point for connective tissue swelling |
| 3. tail | 1 point for the emergence of nodules |
| 4. fore paws | 0.5 points for each paw on which at least one |
| | inflammation occurs on a joint |
| 5. hind paws | 1 point for slight inflammation (swelling) |
| | 2 points for a medium-strength inflammation |
| | 3 points for a massive inflammatory reaction |

On the first day, animals of an “arthritis control” control group were given a subcutaneous injection, into the tail root, with complete Freund's adjuvant but were given only the solvent (i.e., 1% cabrboxymethylcellulose in water). Six animals in each case were used per dose and in the control group. Untreated animals were employed as a further “healthy control” control group. The activity criteria used were the reduction of the increase in the paw volume and the decrease in the arthritis index, compared with the untreated control group, and the weight of the animals, in each case in percent and based on the arthritis control.

In the following table, N-(4-trifluoromethylphenyl)-2-cyano-3-hydroxycrotonamide is compound 1. Colestyramine was administered 4 hours later than compound 1. Table 1 shows the results obtained.

TABLE 1

| | Active | Paw | Arthritis | |
|---|---|---|---|---|
| | substance(mg/kg | Volume | index | Weight |
| | of live weight) | (%) | (%) | (%) |
| Healthy control | | | | 30 |
| Arthritis control | | | | 18 |
| Colestyramine | 1000 | 35 | 44 | 8 |
| Compound 1 | 2.5 | −63 | −77 | 20 |
| Compound 1 + | 2.5 + 1000 | −70 | −92 | 20 |
| Colestyramine | | | | |
| Compound 1 | 7.5 | −83 | −92 | 18 |
| Compound 1 + | 7.5 + 1000 | −73 | −95 | 27 |
| Colestyramine | | | | |
| Compound 1 | 25 | −92 | −100 | −2 |
| Compound 1 + | 25 + 100 | −72 | −100 | 3 |
| Colestyramine | | | | |

Negative values shown in the table indicate a decrease; all other values indicate an increase in comparison with the start of the experiment.

The animals treated with the preparation according to the invention showed a weight increase which, in the case of the amounts 2.5 and 7.5 of compound 1, came very close to the healthy control and was significantly better than with compound 1 alone, while the activity of compound 1 was completely retained.

### EXAMPLE 2

The experimental conditions were analogous to Example 1. The actions of compound 1 and colestyramine on the amount of red blood corpuscles (RBC), hemoglobin content (HGB), hematocrit (HCT), amount of glutamate oxlacetate transaminase (GOT), and glutamate pyruvate transaminase (GPT) were determined. Colestyramine was administered 4 hours later than compound 1. Table 2 shows the results obtained.

TABLE 2

| | | Paw | Arthritis | | | | | | |
|---|---|---|---|---|---|---|---|---|---|
| | Active substance | Volume | index | GOT | GPT | RBC | HGB | HCT | No, of |
| | (mg/kg of live weight) | (%) | (%) | (U/I) | (U/I) | (*10⁶/mm³) | (g/dl) | (%) | animals |
| Healthy | | | | 50.4 | 23.6 | 7.4 | 13.1 | 38.9 | 4 |
| control | | | | | | | | | |
| Arthritis | | | | 50.8 | 20.3 | 7.3 | 13.0 | 38.0 | 6 |
| control | | | | | | | | | |
| Colestyramine | 1000 | 10 | 22 | 43.5 | 25.2 | 7.6 | 10.2 | 31.4 | 6 |
| Compound 1 | 25 | −92 | −100 | 85.3 | 25.1 | 3.8 | 6.1 | 18.7 | 6 |
| Compound 1 + | 25 + 1000 | −72 | −100 | 52.5 | 21.5 | 6.08 | 10.2 | 31.4 | 5 |
| Colestyramine | | | | | | | | | |

Negative values shown in the table indicate a decrease; all other values indicate an increase in comparison with the start of the experiment.

The animals treated with the preparation according to the invention showed a normalization of the amount of red blood corpuscles (RBS), hemoglobin content (HGB), hematocrit (HCT), amount of glutamate oxaloacetate transaminase (GOT), and glutamate pyruvate transaminase (GPT) which came very close to the healthy control and was significantly better than with compound 1 alone. While the activity of compound 1 was completely retained.

### EXAMPLE 3

The experimental conditions were analogous to Example 1. The actions of compound 1 and colestyramine on the amount on the of alkaline phosphatase (AP) and amylase were determined. Colestyramine was administered 4 hours later than compound 1. Table 3 shows the results obtained.

TABLE 3

| | Active | | | | | |
|---|---|---|---|---|---|---|
| | substance | | | | | |
| | (mg/kg of | Paw | Arthritis | | Amy- | No. of |
| | live | volume | Index | AP | lase | tested |
| | weight) | (%) | (%) | (U/I) | (U/I) | animals |
| Healthy | | | | 312.6 | 3058.3 | 6 |
| control | | | | | | |
| Arthritis | | | | 231. | 2251.6 | 6 |
| control | | | | | | |
| Colestyramine | 1000 | −60 | −46 | 271.8 | 2756.6 | 6 |
| Compound 1 | 25 | −110 | −100 | 114.8 | 1306.5 | 6 |
| Compound 1 + | 25 + 1000 | −86 | −94 | 206.6 | 2783.3 | 3 |
| Colestyramine | | | | | | |

Negative values shown in the table indicate a decrease; all other values indicate an increase in comparison with the start of the experiment.

The animals treated with the preparation according to the invention showed a normalization of the amount of alkaline phosphates, which came very close to the healthy control, and was significantly better than with compound 1 alone, while the activity of compound 1 was completely retained.

### EXAMPLE 4

A pharmaceutical composition comprising a preparation according to the invention consists of a small hard gelatin capsule which contains 400 mg of colestyramine and a larger hard gelatin capsule which contains 20 mg of N-(4-trifluoromethylphenyl)-5-methylisoxazole-4-carboxamide. The smaller hard gelatin capsule is completely enclosed by the larger capsule. The filling material employed between the two capsules is glucose.

1. A preparation comprising:
1) at least one compound which essentially prevents the enterohepatic circulation of the nucleotide synthesis inhibitors selected from the group consisting of cholestyramine, colestipol, and activated carbon, and
2) at least one nucleotide synthesis inhibitor selected from the group consisting of (2-morpholinoethyl (e)-6-(1,3-dihydro-4-hydroxy-6-methoxy-7-methyl-3-oxoisobenzofuran-5-yl)-4-methyl-4-hexenoate), mizoribine, a compound of formula (i) or (ii) 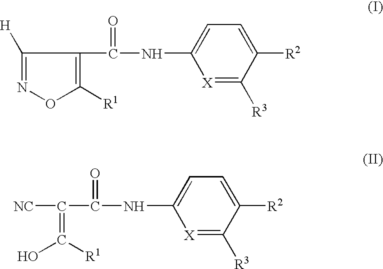
stereoisomeric forms of the compounds of formula (i) or (ii), and a physiologically tolerable salt of the compound of formula (ii), wherein:
R¹ is
a) —(C₁-C₄)-alkyl,
b) —(C₃-C₅)-cycloalkyl,
c) —C₂-C₆)-alkenyl, or
d) —C₂-C₆)-alkynyl;
R² is
a) —CF₃,
b) —O—CF₃
c) —S—CF₃,
d) —OH,
e) —NO₂,
f) halogen,
g) benzyl,
h) phenyl,
i) —O-phenyl, which is unsubstituted,
j) —CN, or
k) —O-phenyl, which is mono- or polysubstituted by
1) —(C₁-C₄)-alkyl,
2) halogen,
3) —O—CF₃, or
4) —O—CH₃;
R³ is
a) —(C₁-C₄)-alkyl,
b) halogen, or
c) a hydrogen atom; and
X is
a) a-CH group, or
b) a nitrogen atom.

2. The preparation as claimed in claim 1, where at least one of a compound of formula (I) and (II), a stereoisomeric form thereof, and a salt of a compound of formula (II) is employed, wherein:
R¹ is
a) methyl,
b) cyclopropyl, or
c) —(C₃-C₅)-alkynyl;
R² is —CF₃, or —CN;
R³ is a hydrogen atom, or methyl, and
X is a —CH group.

3. The preparation as claimed in claim 1, wherein N-(4-trifluoromethyl-phenyl)-5-methylisoxazole-4-carboxamide is a compound of formula (I), or N-(4-trifluoromethyl)-2-cyano-3-hydroxy crotonamide, 2-cyano-3-cyclopropyl-3-hydroxyacrylic acid (4-cyanophenyl)amide, and N-(4-trifluoromethyl-phenyl)-2-cyano-3-hydroxyhept-2-en-6-ynecarboxamide are compounds of formula (II).

4. The preparation as claimed in claim 1, wherein the compound employed which essentially prevents the enterohepatic circulation of the compound of formula (I) or (II) is colestyramine.

5. The preparation as in claim 1, further comprising at least one additional active compound selected from antiuricopathics, analgesics, steroidal or nonsteroidal antiinflammatories, cytokines, cytokine agonists, platelet aggregation inhibitors, cytokine antagonists, and immunosuppressant compounds.

6. The preparation as claimed in claim 5, wherein at least one immunosuppressant compound is selected from cyclosporine A, FK 506, or rapamycin.

7. A pharmaceutical composition, comprising a preparation as claimed in claim 1, wherein the preparation contains at least one nucleotide synthesis inhibitor selected from brequinar, mycophenolatemofetil (2-morpholinoethyl (E)-6-(1,3-dihydro-4-hydroxy-6-methoxy-7-methyl-3-oxoisobenzofuran-5-yl)₄-methyl-4-hexenoate), methotrexatem mizoribine, and compounds of formula (I) and (II); and a compound which essentially prevents the enterohepatic circulation of the compound of formual (I) and (II), or a compound which antagonizes the action of at least one of the nucleotide synthesis inhibitors with a displacement in time, with a pharmaceutically suitable and ohysiologically acceptable vehicle.

8. The pharmaceutical composition as claimed in claim 7, further comprising at least one additional suitable active compound, additive, or excipient.

9. The preparation according to claim 1, wherein said at least one compound which essentially prevents the enterohepatic circulation of the nucleotide synthesis inhibitors is colestyramine, and wherein said at least one nucleotide synthesis inhibitor is N-(4-trifluoromethylphenyl)-2-cyano-3-hydroxycrotonamide.

10. The preparation according to claim 1, wherein said at least one compound which essentially prevents the enterohepatic circulation of the nucleotide synthesis inhibitors is colestyramine, and wherein said at least one nucleotide synthesis inhibitor is N-(4-trifluoromethylphenyl)-5-methylisoxazole-4-carboxamide.

11. The preparation according to claim 1, wherein said at least one compound which essentially prevents the enterohepatic circulation of the nucleotide synthesis inhibitors is colestyramine, and wherein said at least one nucleotide synthesis inhibitor is 2-cyano-3-cyclopropyl-3-hyroxyacrylic acid (4-cyanophenyl)amide.

12. The preparation according to claim 1, wherein said at least one compound which essentially prevents the enterohepatic circulation of the nucleotide synthesis inhibitors is colestyramine, and wherein said at least one nucleotide synthesis inhibitor is N-(4-trifluoromethylphenyl)-2-cyano-3-hydroxyhept-2-en-6-ynecarboxamide.

13. The composition according to claim 7, wherein said composition is a dosage form.

14. The composition according to claim 13, wherein said dosage form is a capsule.
